(19) United States
(12) Patent Application Publication
(10) Pub. No.: US 2006/0127485 A1
(43) Pub. Date: Jun. 15, 2006

(54) Bio-degradable colloid particles, in particular for pulmonary applications

(76) Inventors: Werner Seeger, Biebertal (DE); Thomas Schmehl, Giessen (DE); Tobias Gessler, Wettemberg (DE); Lea Ann Dailey, London (GB); Matthias Wittmar, Medebach (DE)

Correspondence Address: CLARK & BRODY, 1090 VERMONT AVENUE, NW, SUITE 250, WASHINGTON, DC, 20005, US

(21) Appl. No.: 10/534,885

(22) PCT Filed: Nov. 14, 2003
(86) PCT No.: PCT/DE03/03787

§ 371 Date: Dec. 19, 2005

(30) Foreign Application Priority Data
Nov. 15, 2002 (DE) 102 53 623.6

(51) Int. Cl. A61K 9/14; A61K 31/557

(52) U.S. Cl. 424/489; 514/573

(57) ABSTRACT

The invention concerns a compound formed from a biologically degradable, water-soluble comb polymer based on a polyol backbone with side chains carrying amino groups, and, as a stabilizer, at least one negatively charged organic base, which can be a Lewis or Bronsted base, or its corresponding acid, which can be a Lewis or Bronsted acid, wherein the acid groups of the stabilizers are available in excess or deficiency in relation to the primary, secondary or tertiary amino groups or the basic groups are available in excess or deficiency in relation to the quaternary amino groups of the comb polymers, so that the colloidal particles feature a positive or negative zeta potential. Such colloidal particles are particularly suitable for pulmonary application of acidic or negatively charged pharmaceutically active ingredients.

The invention concerns a compound of a biologically degradable amphiphilic comb polymer based on a polyol backbone with positively charged side chains and an additive, in particular an acidic pharmaceutically active ingredient, for the treatment of diseases in humans and higher mammals.

The success of treatment with pharmaceutically active substances is heavily dependent on the bioavailability of the substances in the organism. The bioavailability is, in turn, closely associated with the respective application method of the active substance. For that reason, new application techniques have been developed in the past few years with the aim of making application as simple and patient-friendly as possible. In a multitude of cases, nanoparticular transport vehicles which can take up active substances and release them at the desired location are relied upon. For that purpose, particle-containing systems with biodegradable carriers are considered to be generally advantageous.

Inhalative application is an important means of application of medicaments and active ingredients for the treatment of lung diseases. Furthermore, inhalative application of medicaments is being increasingly employed for the treatment of systemic diseases, since a multitude of active substances deposited in the alveolar region of the lung are quickly taken up by the blood. For both therapeutic applications, a nanoparticular transport vehicle is advantageous because the active ingredients are released continuously after deposition of the nanoparticles in the lungs, ensuring a constant drug level and reducing the number of necessary inhalations. However, an important prerequisite for the inhalative application of a nanoparticular transport vehicle is stability during nebulization, i.e. the stability of the vehicle in relation to the process of aerosol generation.

Breitenbach and Kissel, Polymer 39, page 3261 (1998) describes, i.e., a method for the production of biodegradable comb polymers which can serve as carrier components for such particles. The synthesis of polymers is carried out starting from a polyvinyl alcohol backbone onto which hydrophobic poly(lactic-co-glycolic acid) side chains (PVA-g-PLGA) are grafted.

As a continuation of the work with these comb polymers, the introduction of sulphobutyl side chains in the PVA-g-PLGA comb polymer is described in DE19839515 and EP1132416, whereby the polymers thus obtained are used as carriers for peptidic active substances, in particular for vaccines. In contrast, it was not possible as of yet to make polymers based on such polyol comb polymers available which are suitable for the transport of negatively charged active substances.

Consequently, the provision of a particle-containing transport system is desirable for negatively charged active substances in particular and preferentially for inhalative application. Hereby, it is advantageous when the particle-containing transport system is stable during nebulization.

The task is solved by biodegradable colloidal particles, which contain
a) amphiphilic comb polymers comprising a water-soluble polyol backbone, hydrophobic side chains and primary, secondary, tertiary or quaternary side chains carrying amino groups and
b) as a stabilizer, at least one negatively charged organic base, which can be a Lewis or Bronsted base, or the corresponding acid thereof, which can be a Lewis or Bronsted acid

wherein

the acidic groups of the stabilizers are available in excess or deficiency in relation to the primary, secondary or tertiary amino groups of the comb polymers or the basic groups are available in excess or deficiency in relation to the quaternary amino groups of the comb polymers so that the colloidal particles feature a positive or negative zeta potential.

Preferred comb polymers possess R groups carrying amino groups of the formula [Singular based on the typo from the German version] (II); (III); (IV) and/or (V) as side chains, wherein the R groups of formula (IV) are particularly preferred.

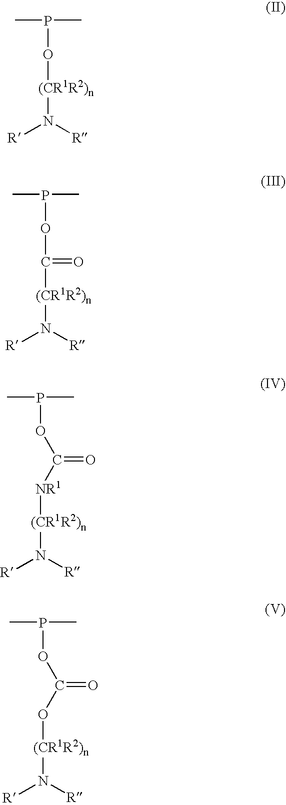

wherein n represents an integer between 1 and 24 and
P stands for the polyol backbone and
R′, R″ stands [the sg. results from a typo in the German version] independently from one another for H, (C₁-C₂₀)-alkyl, (C₂-C₁₀)-alkenyl, (C₂-C₁₀)-alkinyl, (C₆-C₂₂)-aryl alkyl, wherein the R groups R′ and R″ can also be linked to each other and
R¹, R² stands [the sg. results from a typo in the German version] independently from one another for H, (C₁-C₁₀)-alkyl, (C₂-C₁₀)-alkenyl, (C₂-C₁₀)-alkinyl, (C₅-C₁₂)-aryl, (C₆-C₂₂)-alkyl aryl, (C₆-C₂₂)-aryl alkyl, (C₁-C₁₀)-alkoxy, —O(CO)alkyl-(C₁-C₁₀), —(CO)O—alkyl-(C₁-C₁₀), R′R″N-alkyl-(C₁-C₁₀) or —NR′R″,
   wherein also adjacent R groups R¹ or R² can be substituted by double or triple bonds, so that a monounsaturated or polyunsaturated carbon chain is available and
   wherein up to three (CR¹R²) groups can be substituted by an —NR¹—, ═N—, —O— or —(CO)O— group, wherein two ether groups or ester groups are not available adjacently and
wherein also at least two of the R groups R¹, R² can be linked to each other in such a way that a saturated or unsaturated alicyclic, heterocyclic, aromatic or heteroaromatic side chain is available and

wherein the amino groups can be available either completely or partly as quaternary amines of the form —(NRR′R″)⁺, wherein R stands for an H, (C₁-C₂₀)-alkyl, (C₂-C₁₀)-alkenyl, (C₂-C₁₀)-alkinyl or a (C₆-C₂₂)-aryl alkyl R group.

Particularly preferred comb polymers for the production of colloidal particles are, i.e. polymers of the formula (I)

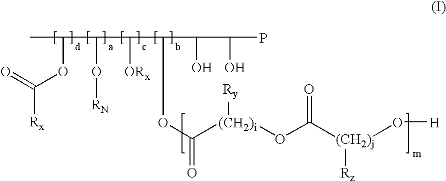

wherein
P represents a polyol backbone,
R_{N} stands for side chains [German version uses plural: side chains, but this was a typo] carrying amino groups according to the formulas (IIa), (IIIa), (IVa) and/or (Va),

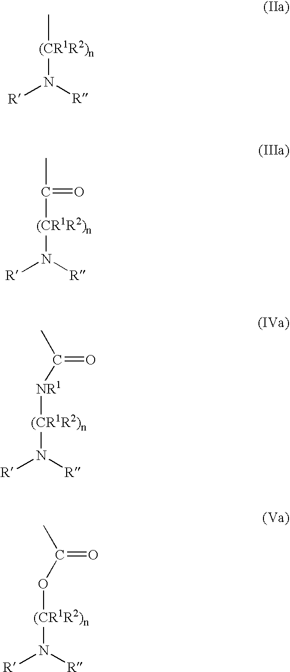

and
Rₓ, R_{y} and R_{z} stands [the sg. results from a typo in the German version] for each of these R groups individually and for each monomer independently from one another for a group chosen from H, (C₁-C₁₀)-alkyl, (C₅-C₁₂)-aryl, (C₆-C₂₂)-alkyl aryl and (C₆-C₂₂)-aryl alkyl and wherein Rₓ can also represent a —(CO)NR′R″ group and wherein R_{y} and R_{z} can also represent a hydroxyl, (C₁-C₁₀)-alkoxy, carboxy, an OCO alkyl-(C₁-C₁₀) or an O(CO)O alkyl-(C₁-C₁₀) group and wherein
i and j stands [the sg. results from a typo in the German version] independently from one another for an integer between 1 and 10 and wherein
m can be an integer between 1 and 100 and
n represents an integer between 1 and 24

and wherein the R groups
R′, R″, R¹, R² possess the aforementioned meaning,

and wherein

the amino groups can be available either completely or partly as quaternary amines of the form —(NRR′R″)⁺, wherein R stands for an H, (C₁-C₂₀)-alkyl, (C₂-C₁₀)-alkenyl, (C₂-C₁₀)-alkinyl or a (C₆-C₂₂)-aryl alkyl R group,

and wherein in relation to the number of the hydroxy functions n_{OH} of the free polyol backbone
a is chosen in such a way that the side chains carrying amino groups possess a percentage of 0.5% to 50%
b is chosen in such a way that the hydrophobic side chains possess a percentage of 1% to 90% and
c, d can be chosen independently from one another in such a way that the ether side chains and ester side chains possess a percentage between 0% and 98.5%, wherein the percentage of these side chains together is less than n_{OH}−(a+b) %.

R groups chosen from the group H, (C₁-C₁₀)-alkyl are particularly preferred for R, R′, R″, R¹ and R². Preferably, the R groups R¹ and R² can also be chosen in such a way that a 5- to 7-membered aromatic or heteroaromatic system, such as a side chain comprising a phenyl group, a naphthyl group, a pyrimidyl group, a pyrolyl group a imidazolyl group, is available.

In particularly preferred comb polymers, the water-soluble polyol backbone P is chosen from the group of polyalcohols, polyvinyl alcohols, polyvinyl acetates, polysaccharides and dextrans.

Particularly preferred comb polymers possess hydrophobic side chains chosen from the group of polylactides, polyglycolides, poly(lactide-co-glycolides), polytartrates, polycaprolactones, poly(lactidic-co-ε-caprolactones), poly(glycolydic-co-ε-caprolactones), or poly(lactidic-co-glycolidic-co-ε-caprolactones).

The proportion of hydrophobic side chains in relation to hydroxy functions of the backbone lies preferably between 20% and 80%, and is particularly preferred to be between 40% and 70%. The proportion of side chains carrying amino groups lies preferably between 2% and 35%, and is particularly preferred to be between 10% and 25%.

Such comb polymers, whether they are available with positively charged side chains (quaternary amino groups) or in their uncharged form, do not build stable colloidal particles on their own, especially not in aqueous solutions. In the presence of organic solvents which are miscible with water, as they are used in the production of colloidal particles, as well as after the removal of the organic solvent, the polymers often remain in their dissolved form and do not demonstrate the desired particle formation. Thus, no definable, stable colloidal particles can be produced exclusively with the described comb polymers comprising amino groups.

It was observed, though, that, using the comb polymers described above, very stable colloidal particles could be produced when negatively charged compounds, in particular polyanions, such as dextran sulphate, carboxymethyl cellulose or nucleic acids, are added during the production of the particles. However, the stabilization of the particles with smaller negatively charged molecules is also possible. Hereby, very stable particles in a range of sizes from 50 nm to 1 μm are obtained via an excess of negatively charged stabilizers, whereby the particle size depends upon the stabilizer as well as the excess of negative charges in the particle, through which particles with a diameter of distinctly less than 1 μm can easily be obtained. This opens the way to adjust the size of the particles to a desired dimension. As a rule, smaller particles of 50 nm to 500 nm, in particular 100 to 250 nm, which can take up enough of the active substance and simultaneously, particularly with regard to the inhalative uptake of the active substance, allow a fine dispersion, are preferred for pharmaceutical applications. The colloidal particles obtained in this way are very stable, which is surprising, particularly with regard to the marginal affinity of pure comb polymers to form colloids.

The excess or deficiency of negative charges which is necessary to generate stable colloidal particles can be determined through determination of the zeta potential of the particles. Hereby, depending on the stabilizer utilized, zeta potentials between −5 and −80 mV or between +5 and +80 mV are suitable. Zeta potentials between −10 and −50 mV or between +10 and +50 mV are preferred. The zeta potential for polyanions, low molecular negatively charged ions and mixtures of polyanions and low molecular substances in particular is especially preferred to be between −20 and −40 mV and between +20 and +40 mV.

As the stabilizer, a pharmaceutically active substance directly, or a combination comprising several pharmaceutically active substances, or a combination of one or more pharmaceutically active substances and an additive is preferably utilized, wherein at least one pharmaceutically active substance is a negatively charged organic base or the corresponding acid thereof. Through the direct utilization of the active substance as the stabilizer, the addition of further additives can be forgone. Simultaneously, the intake capacity as regards the active substance is increased by this.

Acidic pharmaceutically active substances are particularly suitable substances. Particularly preferred are pharmaceutically active substances chosen from the group of carboxylic acids, sulphonic acids or phosphoric acids, which can be converted into colloidal particles, whereby the amino groups of the polymer are quaternized via an acid-base reaction and the active substance is converted into a negatively charged molecule.

The prostanoids represent an example for one such group of active substances, which include i.e. Iloprost®, an active substance for the treatment of pulmonary hypertension.

Active substances whose acidity is not sufficient or which are utilized directly as a negatively charged molecule in the production of colloidal particles can be manipulated, i.e. together with an acid. The addition of other additives, such as tensides, in the production of colloidal particles is also possible, but not necessary.

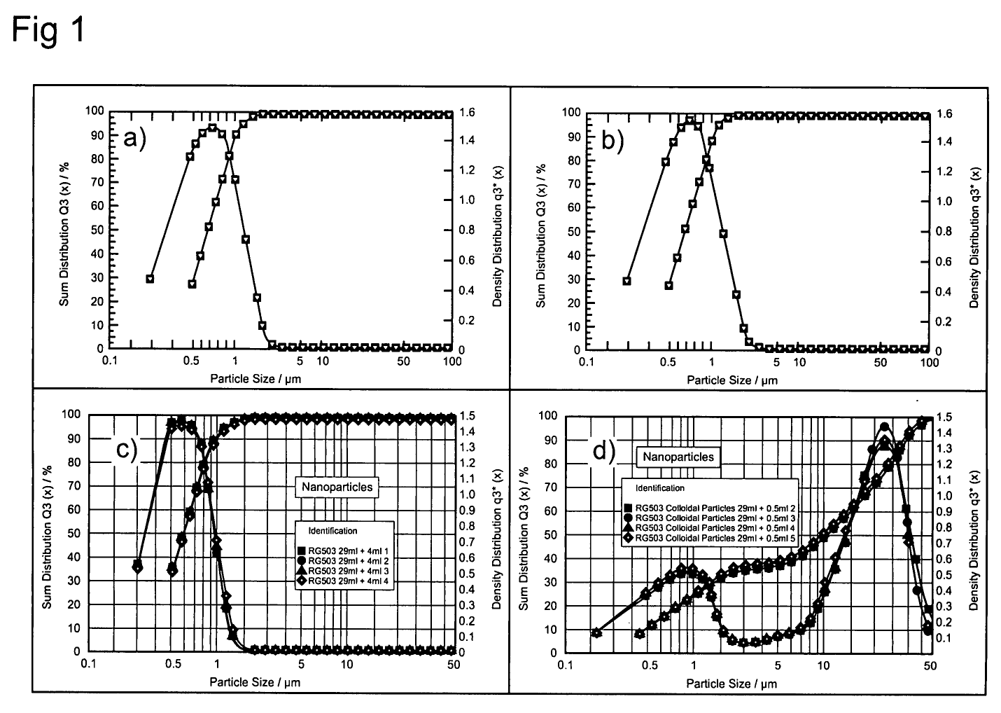

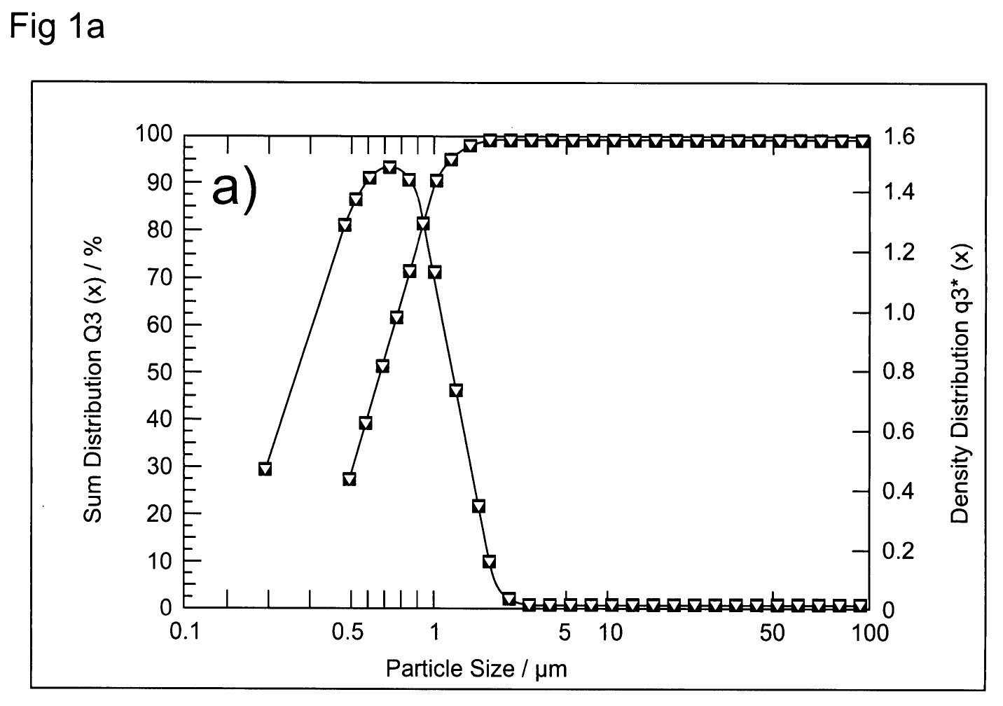

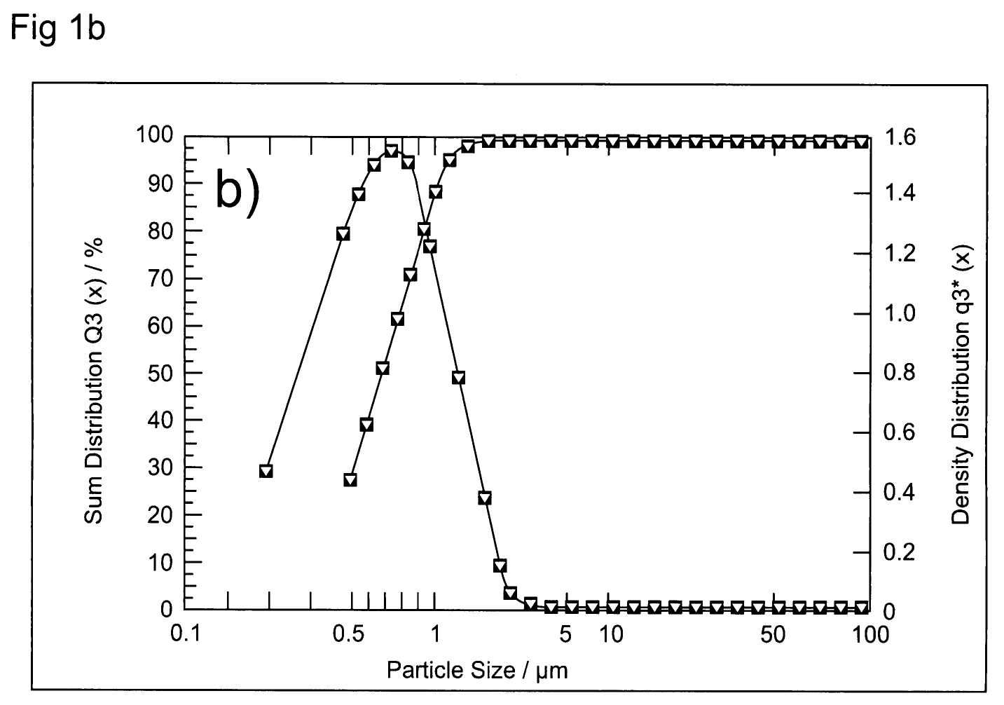

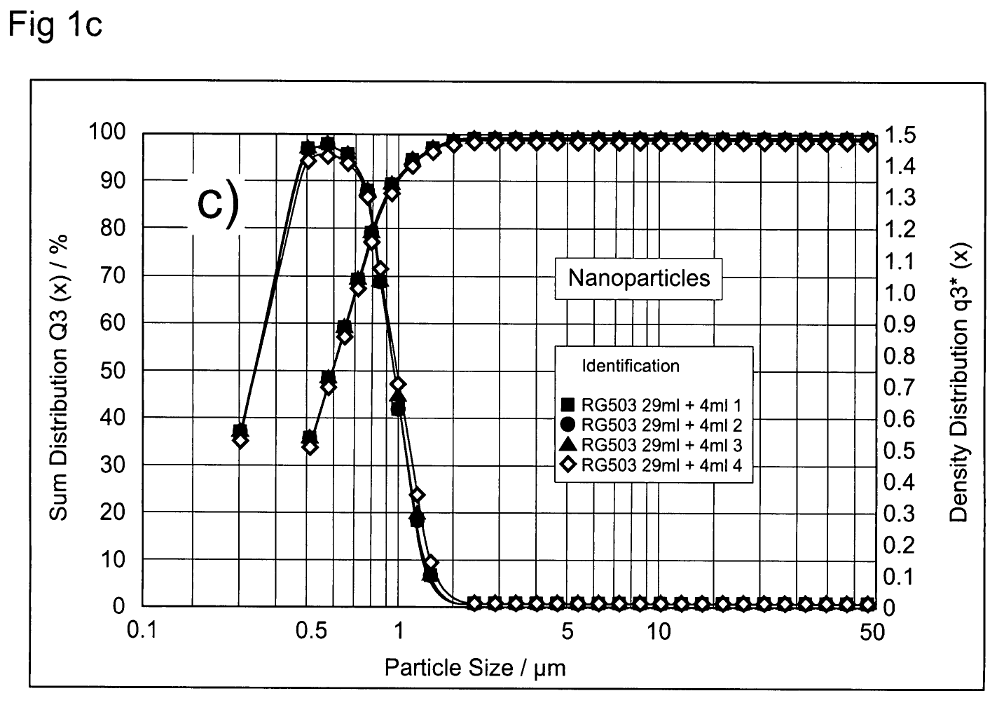

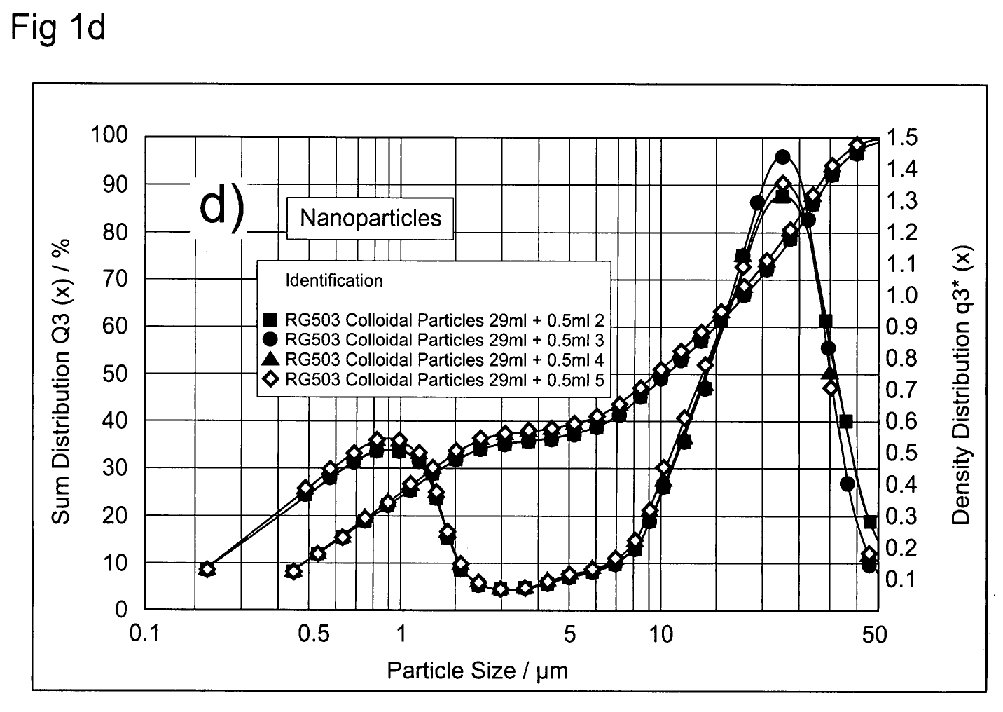

The colloidal particles being claimed are particularly suitable for inhalative application of pharmaceutically active ingredients, since they can be nebulized unchanged from the aqueous phase, as shown in FIG. 1, for example. For this purpose, commercially available nebulizers can be utilized, such as ultrasonic nebulizers, piezoelectric nebulizers, or jet nebulizers. The Pari LC Star (jet nebulizer; Pari Werk GmbH, Starnberg, Germany), the Pulmosonic (ultrasonic nebulizer; DeVilbiss, Langen, Germany), or the Omron U1 (piezoelectric nebulizer; Omron Healthcare GmbH, Hamburg, Germany) are examples of suitable nebulizers which can be utilized.

In order to prevent aggregation of the colloidal particles in such nebulizers, the transported particles, to a high degree, must be stable as regards shear forces, ultrasonic, or mechanical vibrations, since otherwise the practical usability of colloidal suspensions for inhalative applications is heavily impaired.

Furthermore, it can be shown that the particle diameter can be kept within a range of under 1 μm after nebulization without problems, and thus in a range of diameters which is advantageous for deposition in the lungs. Surprisingly, the nebulization stability of the colloidal particles seems to increase with the decreasing hydrophobicity of the comb polymer.

In contrast to this, many conventional colloids form a high proportion of particle aggregates with a diameter of over 5 μm in the nebulization process, through which an efficient dispersal of the aerosol in the lungs is no longer possible.

In addition, the uptake of a high amount of the active substance through inhalation and the fine dispersal of the active substance of the inhaled particles in the lungs is guaranteed through the nebulization stability of colloidal particles with a diameter of less than 1 μm. Consequently, the bioavailability of an inhaled active substance can be considerably increased through the utilization of the particles according to the present invention.

For the reasons mentioned, the simple and patient-friendly inhalative application of the active substance over the alveolar region of the lungs is made accessible through utilization of the colloidal particles being claimed.

Furthermore, the particles are slowly degraded biologically, guaranteeing a continuous release of the active substance over a long period of time. Thus, the particles being claimed are suitable notably as transport vehicles for the treatment of pulmonary and systemic diseases of humans as well as of higher animals.

Positively charged colloidal particles are particularly suitable for mucosal application of pharmaceutically active substances, since they remain adhered to the mucosae extremely well and can be taken up through adsorptive endocytosis. The usability of the particles is not limited only to the mentioned applications, however, as other forms of administration can also be chosen, such as a sublingual, buccal, oral, nasal, vaginal, ocular, or gastrointestinal application.

The colloidal particles according to the present invention can be extracted, i.e., according to the following method:
a) dissolution of a comb polymer comprising a water-soluble polyol backbone, hydrophobic side chains and side chains carrying primary, secondary, tertiary or quaternary amino groups in a water-miscible, volatile organic solvent and
b) addition of the solution obtained in a) to an isotonic aqueous solution with a pH value between 6.0 and 8.0 comprising, along with a sugar and a buffer, an organic acid, which can be a Lewis or Bronsted acid, or the corresponding base thereof, which can be a Lewis or Bronsted base,
c) stirring of the solution obtained in (b) for the production of colloidal particles and
d) removal of the organic solvent.

Biocompatible sugars and buffers, such as glucose and Tris-EDTA buffer, can be utilized in the production of the isotonic solution.

For the production of comb polymers, as described in Breitenbach and Kissel, Polymer 39, page 3261 (1998), or EP1132416, a polyol backbone can be grafted with hydrophobic side chains. The hydrophobicity of the resulting comb polymers can be adjusted during grafting (i.e. by means of a melt-grafting process) through the amount of added side chain monomers. For polyvinyl alcohols with approx. 300 hydroxy groups, the addition of lactides/glycolides in a weight ratio of 1:1 to 1:50, preferentially 1:5 to 1:30, has proven itself.

The binding of side chains carrying amino groups usually occurs before grafting with the hydrophobic side chains. For binding of side chains of the formula (IIa), a chloralkyl ammonium chloride, for example, can be reacted with a free hydroxy group of the polyol backbone. Side chains of the formula (IIIa) can be obtained, i.e. through esterification of the corresponding amino acid. The production of polymers with side chains of the formula (Va) can occur by means of a reaction of the alcohol with phosgene and subsequent addition of the polyol.

For the production of comb polymers with a polyol backbone which carry side chains of the formula (IVa), a separate synthesis had to be developed, wherein starting from a diamine of the formula (VI)

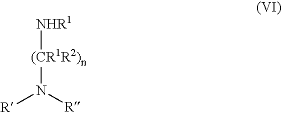

wherein the R groups R′, R″, R1, R2 possess the aforementioned meaning, which is reacted with carbonyldiimidazole, a carbonylimidazole amine, formula (VII)

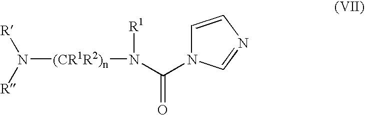

is obtained, which can be linked to a free hydroxy group of the polyol backbone in the presence of an aminopyridine under formation of a urethane linkage.

In the following, the invention is clarified by several practical embodiments, which are not to be regarded as limiting.

### PRACTICAL EMBODIMENT 1: PRODUCTION OF POLYVINYL ALCOHOL COMB POLYMERS WITH SIDE CHAINS CARRYING AMINO GROUPS

1.1. Production of Tertiary Carbonylimidazol Alkylamines

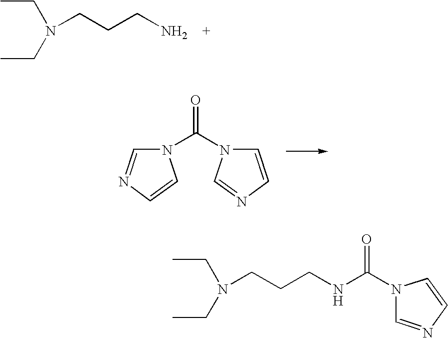

1.62 g (10 mmol) carbonyldiimidazole (CDI) are dissolved in 20 ml of water-free THF, subsequently 10 mmol of an amine (3-dimethylamino propylamine, 3-diethylamino propylamine or 2-diethylamino ethylamine) are added dropwise. The reaction solution is stirred 17 h at room temperature, after which the solvent is evaporated.

Examination of the reaction mixture via NMR yielded [Past tense according to German version] a near 100% conversion.

In a further experiment, a diamine (3-dimethylamino propylamine (DMAPA), 3-diethylamino propylamine (DEAPA), or 2-diethylamino ethylamine (DEAEA)) along with CDI is dissolved in water-free THF. The reaction product is processed analogously to 1.1.

1.2. Synthesis of Modified Polyvinyl Alcohols (PVA)

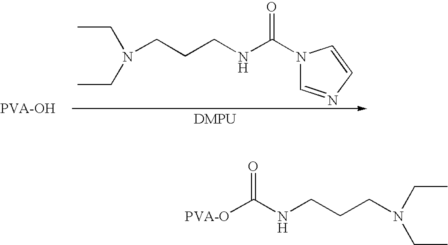

An amount of PVA (polymerisation degree (PD) 300), specified according to Table 1 is dissolved in 170 ml of water-free NMP (N-methylene pyrrolidon) under nitrogen atmosphere. Subsequently, 1,3 dimethyl-3,4,5,6-tetrahydro-2(H1)-pyrimidinone (DMPU) is added. Thereafter, the addition of the amine-carbonyl imidazole (amine-CI) which was produced in 1.1 and taken up in the water-free NMP is carried out. The reaction solution is stirred at 80° C. for 4 to 6 days. The reaction mixture is subsequently ultrafiltrated and the polymer obtained is lyophilized and stored under vacuum at 43° C. for further treatment. [Full stop is missing in German original.]

A qualitative analysis of the reaction mixture is carried out by means of FT-IR (Nicolet FT-IR 510P). For that purpose, the polymer is pressed in pellet form with KBr. Proof of substitution of the polyvinyl alcohol backbone is carried out by means of the signal of the urethane group at 1696 cm⁻¹.

For the determination of the proportion of the side chains carrying amino groups, the polymers thus obtained are dissolved in d₆-DMSO and measured by means of ¹H-NMR (Joel Eclipse 500; GX4000D). For the quantitative analysis, the signals of the amino group were utilized at δ=2.99, 2.21, 2.12, 1.53 (DMAPA) δ=6.89; 2.99; 2.42; 2.36; 0.94 (DEAPA).

The reaction mixtures and results are listed in Table [Sg. results from the typo included in the German version.] 1 and 2.

TABLE 1

| | |
|---|---|
| | Percentage of |
| | amino |
| Embodiment | substituents |

Amine utilized: 3-dimethylamino propylamine (DMAPA)

| | | |
|---|---|---|
| 1 | 12.00 g PVAL, 1.20 g amine-Cl, 0.07 g DMPU, | 2.3% |
| | 200 ml NMP | |
| 2 | 12.00 g PVAL, 2.40 g amine-Cl, 0.16 g DMPU, | 4.4% |
| | 200 ml NMP | |
| 3 | 12.00 g PVAL, 4.80 g amine-Cl, 0.31 g DMPU, | 7.1% |
| | 170 ml NMP | |
| 4 | 12.00 g PVAL, 11.99 g amine-Cl, 0.78 g DMPU, | 10.8% |
| | 170 ml NMP | |
| 5 | 11.00 g PVAL, 23.08 g amine-Cl, 1.51 g DMPU, | 23.0% |
| | 170 ml NMP | |

Amine utilized: 3-diethylamino propylamine (DEAPA)

| | | |
|---|---|---|
| 6 | 10.00 g PVAL, 1.00 g amine-Cl, 0.07 g DMPU, | 2.1% |
| | 200 ml NMP | |
| 7 | 10.00 g PVAL, 2.01 g amine-Cl, 0.06 g DMPU, | 4.0% |
| | 170 ml NMP | |
| 8 | 10.00 g PVAL, 4.02 g amine-Cl, 0.11 g DMPU, | 5.9% |
| | 170 ml NMP | |
| 9 | 10.00 g PVAL, 10.05 g amine-Cl, 0.23 g DMPU, | 10.9% |
| | 120 ml NMP | |
| 10 | 10.00 g PVAL, 23.12 g amine-Cl, 1.32 g DMPU, | 22.7% |
| | 170 ml NMP | |

For the inspection of the results obtained by means of NMR, 5 mg of the polymer thus obtained are analyzed thermogravimetrically with a TGA7 (Perkin Elmer) under nitrogen atmosphere at 20° C. under vacuum. The results are summarized in Table 2.

TABLE 2

| | | | Mass loss | Mass |
|---|---|---|---|---|
| | | Number of | determined with | loss |
| | Molecular | side chains | TGA after the first | expected |
| | weight | carring | decomposition | according |
| Polymer | from | amino groups | step | to |
| (PD 300) | ¹H-NMR) | per polymer | (250-400° C.) | ¹H-NMR |
| 1 | 15318.14 | 2.25% | 4.81% | 7.59% |
| 2 | 15885.98 | 4.35% | 11.80% | 14.15% |
| 3 | 16754.20 | 7.10% | 20.04% | 21.89% |
| 4 | 17875.22 | 10.76% | 36.94% | 31.10% |
| 5 | 22424.22 | 23.04% | 60.67% | 53.08% |
| 6 | 15461.20 | 2.10 | 9.02 | 8.16 |
| 7 | 16100.74 | 4.00 | 12.81 | 14.93 |
| 8 | 16692.34 | 5.90 | 38.19 | 21.24 |
| 9 | 18406.27 | 10.87 | 40.53 | 35.48 |
| 10 | 2311.77 | 22.70 | 58.47 | 57.04 |

1.3. Graft Polymerization with L-Lactide

0.500 g of the modified PVA backbone from 1.2. is reacted with 5.00 g L-lactide in a round-bottomed flask under nitrogen atmosphere. 0.113 g SnOct₂ are added to the reaction mixture as a catalyst. To initiate polymerization, the flask is dipped into an oil bath which was preheated to 150° C., whereby the reaction solution is stirred continuously by a magnetic stirrer. After 3 h, the oil bath is replaced with a cold water bath; subsequently, acetone is added to the reaction mixture. The polymer solution is precipitated out in 250 ml water/isopropanol. After filtration of the reaction product, the polymer thus obtained is dried in vacuo at ambient temperature. The success of the reaction can be inspected by means of ¹H-NMR.

1.4. Production of Polyvinyl Alcohols Carrying Poly(Lactide-Co-Glycolide) Groups

The amine-modified polymer thus obtained is reacted with a mixture of D,L-lactide and glycolide (1:1). In the embodiments which were carried out, the ratio of the utilized polymer to the lactide/glycolide mixture is in 1:1; 1:2; 1:10 and 1:20 stoichiometric proportions in relation to the free hydroxy groups of the PVA backbone. Approx. 10 mol % Sn(II) 2-ethylhexanoat and SnOct₂ are added to the reaction mixture. After a polymerization time of 3 h at 150° C., the reaction mixture is quickly cooled to ambient temperature. The reaction mixture is taken up in acetone and cleaned by precipitating out with a mixture of isopropanol/water or water. The isolated polymer is dried in vacuo at 20° C.

The success of the reaction was inspected by means of ¹H-NMR; the result is listed, for example, for the polymer reaction mixture 10 of embodiment 1.2 and a PVA:glycolide/lactide mixture of 1:20.
¹H-NMR: δ=5.56-5.03, 2.04-1.54 (PVAL signals) 5.21 (lactide), 4.22, 1.46 (lactide end group), 1.30 (lactidene end group) 4.86 (glycolide), 4.08 (glycolide end group) Integrals: lactide central to end 11:1 Integrals: glycolide to lactide end groups 1.6: 21.8 that yields a ratio of lactide : glycolide in side chains: 56:44.

### PRACTICAL EMBODIMENT 2: PREPARATION OF COLLOIDAL PARTICLES

10 mg of the comb polymer (ca. 8% 3-diethylamino propylamine side chains (DEAPA); PVA to lactide/glycolide 1:20) produced according to practical embodiment 1 is dissolved in approx. 3 ml of acetone. 100 μl Tris-EDTA buffer (low ionic strength 100×), a defined amount of stabilizer (carboxymethyl cellulose (CMC); Tylopur® C 600, Hoechst AG are used as polymeric stabilizers; Rose Bengal is used as a low molecular stabilizer), and 0.5 g glucose is added to 10 ml of ultrapure water. The pH value of the aqueous solution is adjusted to pH 7.0 with HCl/NaOH. The aqueous solution is subsequently subjected to sterile filtration with a nitrocellulose membrane filter with a pore size of 0.22 μm. The polymer-containing solution is injected slowly by means of a 20 G×1½ cannula of a 5 ml syringe, with a speed of approx. 0.3 ml/min, into an aqueous phase, present in a beaker containing a magnetic stirrer. During the injection, the cannula opening is pressed lightly onto the wall of the beaker glass. The resulting suspension is stirred 4 h under slightly reduced pressure until the acetone is removed.

The colloidal solutions produced in this way can be nebulized without modification to the colloidal particles.

The influence of the negatively charged stabilizer on the formation and properties of colloidal particles are listed in Tables 3 and 4. The formation of colloids is visually observed, wherein an opalescent mixture indicates the formation of colloidal particles.

TABLE 3

| | | Particle | Zeta potential | |
|---|---|---|---|---|
| Stabilizer | Amount (μg) | diameter (nm) | (mV) | Observation |
| CMC | 2000 | 213.6 ± 3.0 | −46.6 ± 1.1 | opalescent |
| | 1000 | 215.5 ± 2.9 | −28.4 ± 0.4 | opalescent |
| | 500 | 202.5 ± 1.5 | −23.0 ± 0.5 | opalescent |
| | 250 | 202.5 ± 2.9 | −16.6 ± 0.3 | cloudy hazy |
| | 100 | 256.6 ± 4.6 | 39.9 ± 0.3 | cloudy/flaky |
| | 50 | 241.4 ± 1.9 | 45.6 ± 0.7 | cloudy/flaky |
| | 25 | 177.8 ± 1.0 | 47.6 ± 1.7 | opalescent |
| | 0 | 76.2 ± 8.8 | 58.9 ± 1.9 | clear |

TABLE 4

| | | | Particle | Zeta | |
|---|---|---|---|---|---|
| | Amount | Amount | diameter | potential | |
| Stabilizer | (μg) | (μmol) | (nm) | (mV) | Observation |
| Rose | 2840 | 0.26 | 392.8 ± 7.9 | — | cloudy |
| Bengal | 1360 | 0.17 | 246.7 ± 6.1 | −22.2 ± 0.4 | opalescent |
| | 900 | 0.11 | — | — | flaky |

The determination of the size of the colloidal particles is carried out by means of photon correlation spectrometry (PCS). The measurement of the zeta potential is carried out by means of laser Doppler anemometry with the assistance of a Zetasizer 4/AZ 104 (Malvern Instruments, Malvern, UK).

### PRACTICAL EMBODIMENT 3: EXAMINATION OF TEH NEBULIZATION STABILITY

A suspension of CMC comprising colloidal particles according to Practical embodiment 2 is produced.

A suspension of colloidal RG 503 particles (50 mg in 10 ml acetone; Polymer RG 503 Resomer®, Boehringer Ingelheim, Germany) with 0.0025% Alveofact® (Boehringer Ingelheim) is created analogously by means of the Solvent-Displacement-Method (Jung T., et al. J. Controlled Release 67, 157-169, (2000)).

The suspensions are nebulized with a commercial jet nebulizer (Pari LC Star) applying a compressed air flow of 20 l/min through the nebulizer. The aerosol is collected with a clean glass plate. 1 ml of the collected aerosol is subsequently taken up in 29 ml of distilled water. To characterize the nebulized aerosol, the suspensions of CMC comprising colloidal particles which were taken up and, for comparison, the colloidal RG 503 particles are measured with a laser diffractometer (Sympatec GmbH,Claustal-Zellerfeld, Germany). The results are depicted in FIG. 1.

When interpreting the measurement data which was obtained by means of laser diffraction, it should be noted that the method of measurement utilized is very suitable for determining changes in the particle size distribution. The determination of the absolute value for the average diameter of smaller particles (smaller than 1 μm) can be carried out considerably more precise when using photon correlation spectroscopy (PCS). The corresponding values are listed in Practical embodiment 2, Table 3.

As shown in FIG. 1, the particles according to the present invention are characterized by an uncommonly high nebulization stability. The particle size distribution before (FIG. 1a) and after (FIG. 1b) nebulization remained virtually unchanged. RG 503 features a similar particle size distribution in its non-nebulized state as the claimed particles. After nebulization however, RG 503 features a strong affinity towards aggregation (FIG. 1d), whereby the proportion of particles with a larger diameter increases significantly.

1. Biodegradable colloidal particles comprising
a) amphiphilic comb polymers comprising a water-soluble polyol backbone, hydrophobic side chains and primary, secondary, tertiary or quaternary side chains carrying amino groups and
b) as a stabilizer, at least one negatively charged organic base, which can be a Lewis or Bronsted base, or the corresponding acid thereof, which can be a Lewis or Bronsted acid
wherein
the acid groups of the stabilizer are available in excess or deficiency in relation to the primary, secondary, or tertiary amino groups of the comb polymers, or
the basic groups are available in deficiency or excess in relation to the quaternary amino groups of the comb polymers,
so that the colloidal particles feature a positive or negative zeta potential.

2. Biodegradable colloidal particles according to claim 1, wherein the organic base or corresponding acid thereof is a pharmaceutically active substance, or a combination comprising several pharmaceutically active substances, or a combination of one or more pharmaceutically active substances and an additive.

3. Biodegradable colloidal particles according to claim 2, wherein the pharmaceutically active substance is a carboxylic acid, sulphonic acid or phosphoric acid.

4. Biodegradable colloidal particles according to claim 3, wherein the active substance is a prostanoid.

5. Biodegradable colloidal particles according to claim 3, wherein the active substance is Iloprost®.

6. Biodegradable colloidal particles according claim 1, wherein the side chains carrying amino groups are R groups of the formula [Sg. results from a typo in the German version] (II), (III), (IV), and/or (V), 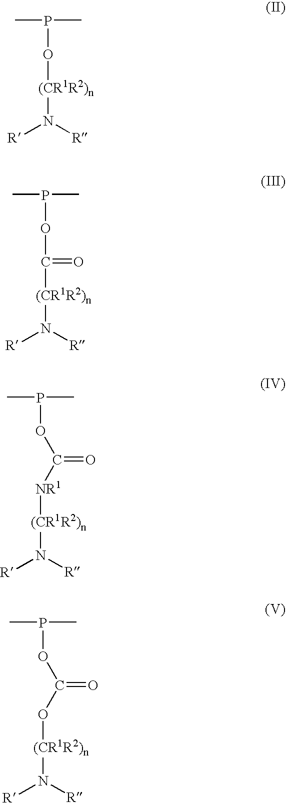
wherein n represents an integer between 1 and 24 and
P stands for the polyol backbone and
R′, R″ stands [the sg. results from a typo in the German version] independently from one another for H, (C₁-C₂₀)-alkyl, (C₂-C₁₀)-alkenyl, (C₂-C₁₀)-alkinyl, (C₆-C₂₂)-aryl alkyl, wherein the R groups R′ and R″ can also be linked to each other and
R¹, R² stands [the sg. results from a typo in the German version] independently from one another for H, (C₁-C₁₀)-alkyl, (C₂-C₁₀)-alkenyl, (C₂-C₁₀)-alkinyl, (C₅-C₁₂)-aryl, (C₆-C₂₂)-alkyl aryl, (C₆-C₂₂)-aryl alkyl, (C₁-C₁₀)-alkoxy, —O(CO)alkyl-(C₁-C₁₀), —(CO)O-alkyl-(C₁-C₁₀), R′R″N-alkyl-(C₁-C₁₀) or —NR′R″,
wherein also adjacent R groups R¹ or R² can be substituted by double or triple bonds, so that a monounsaturated or polyunsaturated carbon chain is available and
wherein up to three (CR¹R²) groups can be substituted by an —NR¹—, ═N—, —O— or —(CO)O— group, wherein two ether groups or ester groups are not available adjacently and
wherein also at least two of the R groups R¹, R² can be linked to each other in such a way that a saturated or unsaturated alicyclic, heterocyclic, aromatic or heteroaromatic side chain is available and
wherein the amino groups can be available either completely or partly as quaternary amines of the form —(NRR′R″)⁺, wherein R stands for an H, (C₁-C₂₀)-alkyl, (C₂-C₁₀)-alkenyl, (C₂-C₁₀)-alkinyl or a (C₆-C₂₂)-aryl alkyl R group.

7. Biodegradable colloidal particles according to claim 1, wherein the comb polymer features a water-soluble polyol backbone chosen from the group of polyalcohols, polyvinyl alcohols, polyvinyl acetates, polysaccharides and dextrans.

8. Biodegradable colloidal particles according to claim 1, wherein the comb polymer features hydrophobic side chains chosen from the group of polylactides, polyglycolides, poly(lactide-co-glycolides), polytartrates, polycaprolactones, poly(glycolydic-co-ε-caprolactones), poly(lactidic-co-ε-caprolactones), or poly(lactidic-co-glycolidic-co-ε-caprolactones).

9. Biodegradable colloidal particles according to claim 1, wherein 0.5 to 50% of the hydroxy groups of the polyol backbone of the comb polymer possess linked side chains carrying amino groups and 1 to 90% of the hydroxy groups of the polyol backbone possess linked hydrophobic side chains.

10. Biodegradable colloidal particles according to claim 1, wherein the amino groups of the comb polymer are at least partly quaternary amino groups, which were quaternized via the addition of an organic acid and stabilized via the corresponding negatively charged organic base which resulted from this.

11. Biodegradable colloidal particles according to claim 1, wherein the zeta potential lies between −5 and −80 mV or between +5 and +80 mV.

12. Biodegradable colloidal particles according to claim 1, wherein the organic acid is a prostanoid and the zeta potential lies between −10 and −50 mV or between +10 and +50 mV.

13. Utilization of biodegradable colloidal particles according to claim 1 for the application of an acidic or basic pharmaceutically active substance for the treatment of diseases in humans and mammals.

14. Utilization of biodegradable colloidal particles according to claim 1 for the inhalative (pulmonary) application of an acidic or basic active substance.

15. Utilization of biodegradable colloidal particles according to claim 13, wherein the pharmaceutically active substance is a prostanoid.

16. Utilization of biodegradable colloidal particles according to claim 13 for the treatment of pulmonary hypertension.

17. Utilization according to claim 13, wherein the biodegradable colloidal particles are available in a physiologically compatible aqueous solution.

18. Method for the production of biodegradable colloidal particles according to claim 1 encompassing the following steps for production.
a) dissolution of a comb polymer comprising a water-soluble polyol backbone, hydrophobic side chains and side chains carrying primary, secondary, tertiary or quaternary amino groups in a water-miscible, volatile organic solvent and
b) addition of the solution obtained in a) to an isotonic aqueous solution with a pH value between 6.0 and 8.0 comprising, along with a sugar and a buffer, an organic acid, which can be a Lewis or Bronsted acid, or the corresponding base thereof, which can be a Lewis or Bronsted base,
c) stirring of the solution obtained in (b) for the production of colloidal particles and
d) removal of the organic solvent.

19. Comb polymer of the general formula (I), 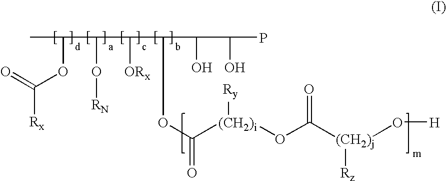
wherein
P represents a polyol backbone,
R_{N} stands for a side chain carrying amino groups according to the formulas (IIa), (IIIa), (IVa) and/or (Va), 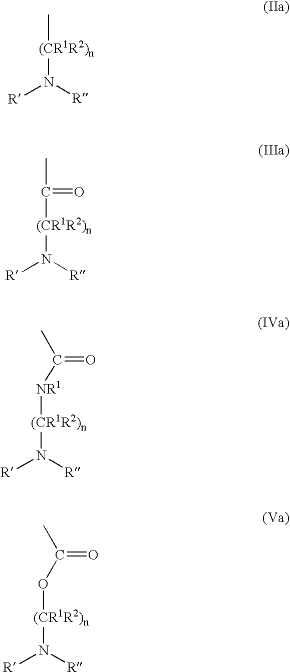
and
Rₓ, R_{y} and R_{z} stands [the sg. results from a typo in the German version] for each of these R groups individually and for each monomer independently from one another for a group chosen from H, (C₁-C₁₀)-alkyl, (C₅-C₁₂)-aryl, (C₆-C₂₂)-alkyl aryl and (C₆-C₂₂)-aryl alkyl and wherein Rₓ can also represent a —(CO)NR′R″ group and wherein R_{y} and R_{z} can also represent a hydroxyl, (C₁-C₁₀)-alkoxy, carboxy, an OCO alkyl-(C₁-C₁₀) or an O(CO)O alkyl-(C₁-C₁₀) group and wherein
i and j stands [the sg. results from a typo in the German version] independently from one another for an integer between 1 and 10 and wherein
m can be an integer between 1 and 100
and wherein
R¹, R², R′, R″ possess the meaning mentioned in claim 6.
and wherein
the amino groups can be available either completely or partly as quaternary amines of the form —(NRR′R″)⁺, wherein R stands for an H, (C₁-C₂₀)-alkyl, (C₂-C₁₀)-alkenyl, (C₂-C₁₀)-alkinyl or a (C₆-C₂₂)-aryl alkyl R group,
and wherein, relating to the number of the hydroxy functions n_{OH} of the free polyol backbone
a is chosen in such a way that the side chains carrying amino groups possess a percentage of 0.5% to 50%
b is chosen in such a way that the hydrophobic side chains possess a percentage of 1% to 90% and
c, d can be chosen independently from one another in such a way that the ether side chains and ester side chains possess a percentage between 0% and 98.5%, wherein the percentage of these side chains together is less than n_{OH}−(a+b) %.

20. Comb polymer according to claim 19, wherein
a is chosen in such a way that the side chains carrying amino groups possess a percentage of 2% to 35% and
b is chosen in such a way that the hydrophobic side chains possess a percentage of 40% to 70%.

21. Comb polymer according to claim 19, wherein the hydrophobic side chains are chosen from polylactide, polyglycolide, poly(lactide-co-glycolide), polytartrate, polycaprolactone, poly(lactidic-co-ε-caprolactone), poly(glycolydic-co-ε-caprolactone), or poly(lactidic-co-glycolidic-co-ε-caprolactone) R groups.

22. Method for the modification of comb polymers with a polyol backbone with side chains of the formula (IV) according to claim 6, wherein a diamine of the formula (VI), 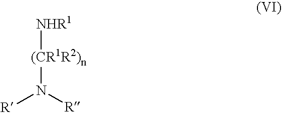
wherein the R groups R′, R″, R¹, R² have the meaning stipulated in claim 6, is reacted with carbonyidiimidazole, and the resulting carbonylimidazole amine (formula (VII)) 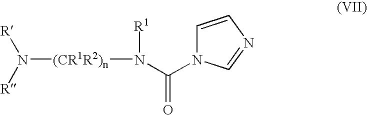
is linked to a free hydroxy group of the polyol backbone in the presence of an aminopyridine under formation of a urethane linkage.

23. Utilization of comb polymers according to claim 19 for the production of biodegradable colloidal particles.

24. The utilization of comb polymers, wherein the biodegradable colloidal particles of claim 23 comprise:
a) amphiphilic comb polymers comprising a water-soluble polyol backbone, hydrophobic side chains and primary, secondary, tertiary or quaternary side chains carrying amino groups and
b) as a stabilizer, at least one negatively charged organic base, which can be a Lewis or Bronsted base, or the corresponding acid thereof, which can be a Lewis or Bronsted acid
wherein
the acid groups of the stabilizer are available in excess or deficiency in relation to the primary, secondary, or tertiary amino groups of the comb polymers, or
the basic groups are available in deficiency or excess in relation to the quaternary amino groups of the comb polymers,
so that the colloidal particles feature a positive or negative zeta potential.
